(19) United States
(12) Patent Application Publication
(10) Pub. No.: US 2009/0012564 A1
(43) Pub. Date: Jan. 8, 2009

(54) TRANSDISCAL INTERBODY FUSION DEVICE AND METHOD

(75) Inventors: Paul E. Chirico, Campbell, CA (US); Benny M. Chan, Fremont, CA (US); R. Sean Pakbaz, San Diego, CA (US); Joseph A. Horton, Hoover, AL (US); Alison M. Souza, Santa Clara, CA (US); Brian E. Martini, Aptos, CA (US)

Correspondence Address: SHAY GLENN LLP, 2755 CAMPUS DRIVE, SUITE 210, SAN MATEO, CA, 94403, US

(73) Assignee: SPINEWORKS MEDICAL, INC., San Jose, CA (US)

(21) Appl. No.: 12/044,884

(22) Filed: Mar. 7, 2008

Related U.S. Application Data
(60) Provisional application No. 60/905,506, filed on Mar. 7, 2007.

(51) Int. Cl. A61B 17/70; A61B 19/00; A61B 17/04; A61F 11/00

(52) U.S. Cl. 606/246; 128/898; 606/327; 606/108

(57) ABSTRACT

Described herein are devices and systems for transdiscal fusion of vertebrae and methods for fusing adjacent vertebra. A system may include a device with two anchorable members connectable by an intervening connector forming a continuous passageway therethrough. An anchorable member may have a constrained non-anchoring configuration and a released anchoring configuration. The anchoring configuration typically includes a radially-expanded structure such as a plurality of struts. After positioning the anchorable members into two adjacent vertebral bodies, the anchorable members may be released from their constrained configuration so that they radially self-expand, anchoring the device across the fracture. A flowable bone-filling material may be conveyed into the passageway of the device after implantation, stabilizing it further in the vertebral implantation site.

### CROSS REFERENCE TO RELATED APPLICATIONS

This application claims priority to U.S. Provisional Patent Application Ser. No. 60/905,506, entitled “Transdiscal Interbody Fusion Device and Method”, filed on Mar. 7, 2007.

### FIELD OF THE INVENTION

The invention relates to a system and methods for using the system to treat bone within a skeletal structure, more particularly to vertebral bodies.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND OF THE INVENTION

Osteoporosis, a disease of bone tissue that is characterized by bone micro-architecture deterioration and loss of bone mass, leads to bone fragility and an increase fracture risk. Vertebral compression fractures consequent to osteoporotic degeneration have serious consequences, with patients suffering from loss of height, deformity, and persistent pain that can significantly impair mobility and quality of life. An estimated 1.5 million elderly people in the United States suffer an osteoporotic fracture each year, with women being at higher risk than men. In addition to the consequences specific to each individual vertebra, compromise of a series of vertebra can create misalignment between the individual vertebrae and cause an even more difficult condition for an afflicted patient, with associated pain and loss of mobility. The lumbar region of the spine is most commonly affected by degenerative disease and consequent misalignment, but thoracic and cervical regions may also be affected.

One surgical approach to treating spinal degeneration and loss of proper alignment is spinal fusion, where adjacent individual vertebrae are joined together. There are two main types of lumbar spinal fusion, which may be used in conjunction with each other, postlateral fusion and interbody. Posterolateral fusion places grafted bone between the transverse processes in the back of the spine. These vertebrae are then fixed in place with hardware through the pedicles of each vertebra that attaches to a metal rod on each side of the vertebrae. Interbody fusion places grafted bone between the vertebra in the area normally occupied by the intervertebral disc, but which has been removed. A device may be placed between the vertebrae to maintain spine alignment and disc height.

In most cases, the fusion is augmented by a process called fixation, meaning the placement of metallic screws (pedicle screws often made from titanium), rods or plates, or cages to stabilize the vertebra to facilitate bone fusion. The fusion process typically takes 6-12 months after surgery. During in this time external bracing (orthotics) may be required. Some newer technologies have been introduced which avoid fusion and preserve spinal motion. Such procedures, such as artificial disc replacement are being offered as alternatives to fusion, but have not yet been adopted on a widespread basis in the US. Newer approaches to vertebral body fusion that reduce the amount, area, or frequency of surgical intervention would be very welcome in the surgical spinal care market.

### SUMMARY OF THE INVENTION

Described herein are devices and systems for stabilizing two adjacent vertebral bodies and methods of stabilizing adjacent vertebral bodies. In general, these devices include a first anchorable member, a second anchorable member and a connector configured to connect the first and second anchorable members. When the first anchorable member is connected to the second anchorable member by the connector, the device has a central passageway through which a material (e.g., cement) may be delivered. The anchorable members are typically self-expanding.

For example, a method for stabilizing two adjacent vertebral bodies may include forming a disc-traversing channel in adjacent first and second vertebral bodies (e.g., through the adjacent endplate regions), positioning a system for stabilizing the adjacent vertebral bodies in the channel, and anchoring the first anchorable member within the first vertebral body and the second anchorable member within the second vertebral body. The system for stabilizing the vertebral bodies may include a transdiscal intervertebral body fusion device with a first anchorable member and a second anchorable member, each member having a central passageway, each member having a constrained non-anchoring configuration and a released anchoring configuration, and a connector having a central passageway, the connector attachable to the proximal end of the first anchorable member and the distal end of the second anchorable member, such that the central passageways of the anchorable members and the connector form a continuous passageway.

In typical embodiments of the method, the first and second vertebral bodies are aligned into a natural or desired orientation before forming a channel for the implantation of the system. The channel may be formed in at least two ways. One approach includes forming the channel by percutaneously entering the second vertebral body, continuing through the disc, and terminating in the first vertebral body. Another approach includes percutaneously entering the vertebral space between the adjacent vertebral bodies to create an access channel, forming a first portion of the disc traversing channel into the first vertebral body, and then forming a second portion of the disc traversing channel into the second vertebral body, to form a complete channel.

In some embodiments, the method includes inserting an anchorable member into the channel in the constrained configuration. Constraining and releasing the anchorable members may be done in at least two ways. In one approach, the anchorable members are constrained or confined in the constrained configuration by preventing their radial expansion with a sleeve. With these embodiments, releasing the anchorable members to their expanded configuration includes ejecting them from the sleeve. In another approach, the anchorable members are held in the constrained configuration by applying tension across the length of the members, which without constraint would contract along their length. In these embodiments, releasing the anchorable members to their radially expanded configuration includes releasing the tension from across the length of the members.

In some embodiments, anchoring the members in the channel includes radially expanding a plurality of bowed struts from each anchorable member to anchor the first member and the second member within the first and second vertebral bodies respectively. In some embodiments the expansion of the bowed struts is by self-expansion. And in some embodiments, expansion of the bowed struts includes mechanically assisting the expansion after the bowed struts have self-expanded to the extent that they can in situ. Further, in some embodiments, anchoring the members includes exposing bone-cutting surfaces on the leading edge of the expanding bowed struts.

In some embodiments, during the anchoring of the members, the first and second anchorable members expand simultaneously or nearly simultaneously. In other embodiments, the first anchorable member expands first, and the second anchorable member expands.

In some embodiments, anchoring the members includes flowable a material, such as a bone filling composition, through the continuous passageway within the system. Flowing the material through the passageway may include flowing the material through holes in the connector or from other portions of the continuous passageway, which may then flow into space within the expanded members, or into space peripheral to the implanted system, where the material may harden or set.

In some variations, anchoring the system includes making adjustments to the anchorable member (or members) after the bowed struts have self-expanded. One form of adjustment includes mechanically assisting the expansion of the struts, as mentioned above. Another form of adjustment may include drawing the anchorable members closer together. One approach to drawing them together is by rotating the connector which may be threadably engaged with one or both of the anchorable members.

Also described herein are methods of stabilizing adjacent vertebral bodies including the steps of: forming a channel in adjacent first and second vertebral bodies through adjacent endplate regions; anchoring a first anchorable member within the channel in the first vertebral body; anchoring a second anchorable member within the channel in the second vertebral body; and flowing a material through a continuous central passageway formed through the first anchorable member, the second anchorable member and a connector between the first anchorable member and the second anchorable member.

### BRIEF DESCRIPTION OF THE FIGURES

FIGS. 1A-1F show one variation of a device that be used as a transdiscal intervertebral body fusion device or system. This device has a circular cross-section with two expandable members, each with four radially expandable struts. The struts have a flat expanding surface. FIG. 1A is a perspective view of the body of the device. FIG. 1B is a side view of the body of the device showing slots forming the struts. FIG. 1C is a cross-sectional view of the device. FIG. 1D is a perspective view of the device after the struts have radially expanded. FIG. 1E is a side view of the device after the struts have radially expanded. FIG. 1F is an end view of the device after the struts have radially expanded.

FIGS. 2A-2F show an internal-external, or double-bodied, device which may be used as a transdiscal intervertebral body fusion device (or system), wherein each body includes two expandable members (or regions), each with four expandable struts. The struts of the internal and external bodies are staggered with respect to each other. FIG. 2A is a perspective view of the device in the unexpanded (insertion) configuration. FIG. 2B is a side view of the body of the device. FIG. 2C is a cross-sectional view of the device. FIG. 2D is a perspective view of the device after the struts have radially expanded. FIG. 2E is a side view of the device after the struts have radially expanded. FIG. 2F is an end view of the device after the struts have radially expanded.

FIGS. 3A-3F show a device which may be used as a transdiscal intervertebral body fusion device (or system) with a rectangular body and four radially expandable struts, each arising from a cut through a flat surface of the body and expanding with a leading sharp edge. FIG. 3A is a perspective view of the body of the device. FIG. 3B is a side view of the body of the device showing slots forming the struts. FIG. 3C is a cross-sectional view of the device. FIG. 3D is a perspective view of the device after the struts have radially expanded. FIG. 3E is a side view of the device after the struts have radially expanded. FIG. 3F is an end view of the device after the struts have radially expanded.

FIGS. 4A-F shows a device which may be used as a transdiscal intervertebral body fusion device (or system) with a rectangular body and two radially expandable struts arising from length-wise cuts in a flat surface of the body and expanding with a leading flat edge. FIG. 4A is a perspective view of the body of the device. FIG. 4B is a side view of the body of the device showing slots. FIG. 4C is a cross-sectional view of the device. FIG. 4D is a perspective view of the device after the struts have radially expanded. FIG. 4E is a side view of the device after the struts have radially expanded. FIG. 4F is an end view of the device after the struts have radially expanded.

FIGS. 5A-5F show a device which may be used as a transdiscal intervertebral body fusion device (or system) with a rectangular body and two radially expandable struts formed by length-wise cuts at a vertex of the rectangle, each strut expanding with a leading sharp edge. FIG. 5A is a perspective view of the body of the device. FIG. 5B is a side view of the body of the device showing slots to be cut from which struts will emerge. FIG. 5C is a cross-sectional view of the device. FIG. 5D is a perspective view of the device after the struts have radially expanded. FIG. 5E is a side view of the device after the struts have radially expanded. FIG. 5F is an end view of the device after the struts have radially expanded.

FIG. 6 shows a single anchorable member with two radially opposed struts in an expanded configuration, the member being a component joinable with a connector portion and a second anchor to form a complete transdiscal intervertebral body fusion device.

FIG. 7 shows a perspective view of a single anchorable member with three radially distributed struts in an expanded configuration, the member being a component that is joinable with a connector portion and a second anchor to form a complete transdiscal intervertebral body fusion device.

FIG. 8 shows a perspective view of a single anchorable member with four radially opposed struts in an expanded configuration, the member being a component joinable with a connector portion and a second anchor to form a complete transdiscal intervertebral body fusion device, the anchorable member further including a central rod that maintains a continuous passageway with a connector in the fully assembled device. The connector portion and/or rod include holes from which a flowable bone cement may be ejected.

FIGS. 9A and 9B show a device which may form a transdiscal intervertebral body fusion device (or system) with a rectangular body and two radially expandable struts emanating from length-wise cuts at a vertex of the rectangle. This device is similar to that depicted in FIG. 5 except that the corners of the rectangle have been pinched or crimped in, giving the corner an angle more acute than 90 degrees. These acute corners become the leading edge of a strut as it expands, and in this embodiment the leading edge is particularly sharp. FIG. 9A is a perspective view of the body of the device. FIG. 9B is a partial view through an expanded struts.

FIGS. 10A-10F show one variation of an anchorable member which may be used as part of a transdiscal intervertebral body fusion device or system. This variation includes a linearly corrugated surface, from which nine expandable struts emanate. FIG. 10A shows the body of the anchorable anchorable member in a linearly constrained, non-radially expanded configuration. Slots are present though not visible in the inner vertex of corrugations. FIG. 10B shows expansion of the expandable struts to a first position, which may either be a partial or fully self-expanded configuration. FIG. 10C shows expansion of the expandable struts to a second position, more expanded than the first position of FIG. 10B. FIG. 10D shows a linearly cross sectional view at position 10D of FIG. 10A, showing the corrugated nature of the body of the expandable member. FIG. 10E shows a linearly cross sectional view at position 10E of FIG. 10B, showing the M-shaped cross-sectional profile the expanded struts. FIG. 10F shows a linearly cross sectional view at position 10F of FIG. 10C, showing the flattened M-shaped cross-sectional profile the expanded struts.

FIG. 11A shows a device which may be used as a transdiscal intervertebral body fusion device (or system) exploded into three parts, illustrating various dimensions of the device. FIG. 11B shows a cross section of the body of an anchorable member. FIG. 11C shows a cross section of the struts at their most expanded point. FIG. 11D shows a cross section of an alternative embodiment with three struts rather than four struts.

FIGS. 12A-12E show various embodiments of device that may be used as transdiscal intervertebral body fusion devices or systems that have dissimilar first and second anchoring or anchorable members for custom fitting into adjacent vertebral bodies. FIG. 12A is a device with a three-strut anchorable member and a two-strut anchorable member, in each case that struts curvilinear and asymmetrically bowed. FIG. 12B is a device with a two-strut anchorable member and a four-strut anchorable member, the struts on each anchor are symmetrically bowed, have substantially straight segments, and are about the same size. FIG. 12C is a device with a four-strut anchorable member that is significantly larger than its two-strut companion. FIG. 12D is a device with two four-strut anchorable members, both asymmetrically bowed, one anchorable member being larger than the other. FIG. 12E is a device with one three-strut anchorable member and a larger four-strut anchorable member, the struts being symmetrical with substantially straight segments.

FIGS. 13A-13H illustrate the deployment of an integrated transdiscal intervertebral fusion device into two adjacent vertebral bodies through a channel that enters the wall of one of the vertebral bodies, and continues through the disc and terminates in the interior of the adjacent vertebral body, the device having an internal-external double body configuration, each body having four expandable struts.

FIG. 13A shows a delivery device or cannula having entered a caudal vertebral body and penetrated through the disc cephalad to it, and into the cephalad vertebral body.

FIG. 13B shows deployment of the first or distal anchorable member, still in its constrained or linear configuration.

FIG. 13C shows the delivery device having been removed from the cephalad vertebral body and the anchorable member with struts expanded.

FIG. 13D shows the delivery device still further withdrawn, past the disc, and the connector portion of the device now spanning the disc.

FIG. 13E shows the delivery device partially removed from the caudal vertebral body and the proximal or second anchorable member now exposed but prior to expansion of the struts.

FIG. 13F shows the delivery device withdrawn to a point such that the proximal or second anchorable member is released from constraint, and the struts having expanded.

FIG. 13G shows a flowable cement being injected through the delivery device, and the cement emerging from the device into the spaces within and surrounding the anchored members.

FIG. 13H shows the region after removal of the delivery device, and the device transdiscally-implanted, anchored by expanded struts, and stabilized by the injected cement, now hardened in place.

FIGS. 14A-14H illustrates the deployment of a transdiscal intervertebral fusion device into two adjacent vertebral bodies through an intervertebral access channel, from which separate but contiguous cephalad and caudal channels are made into the interior of each and adjacent vertebral body, the device having an internal-external double body configuration, each body having four expandable struts. FIG. 14A shows a cannula-delivered drill entering a vertebral space and creating an entry into the cephalad vertebral body to form a portion of a channel to receive an anchorable member of a transdiscal intervetebral body fusion device.

FIG. 14B shows a cannula-delivered drill entering a vertebral space and creating an entry into the caudal vertebral body to form a portion of a channel to receive an anchorable member of transdiscal intervetebral body fusion device.

FIG. 14C shows the positioning of an anchorable member into the cephalad vertebral body as it is being pushed from a cannula.

FIG. 14D shows the anchorable member self-expanding within the cephalad vertebral body upon full emergence from the cannula.

FIG. 14E shows the insertion of a second anchorable member into the caudal vertebral body; in this embodiment the second anchorable member includes a proximally-directed connector within the anchorable member, which can be engaged and drawn out from the interior.

FIG. 14F shows a tool having engaged the connector and drawn it out of the interior of the second anchorable member, placing it so that it can engage the first anchorable member.

FIG. 14G shows the transdiscal intervertebral body fusion device after a bone filling composition has been injected into the space within the expanded anchor. A gear mounted on the side of the connector is being turned by a complementary gear head at the end of a cable that has been delivered to the site by a cannula, the turning of the gear results ultimately in the drawing together of the first and second anchorable members. FIG. 15 provides a detailed view of this mechanism.

FIG. 14H shows the fully assembled transdiscal intervertebral body fusion device, now positioned by the connector adjustment transdiscal intervertebral body fusion device.

FIG. 15 shows components of a transdiscal intervertebral body fusion kit, the kit including an Allen head tool, a first and a second anchorable member, two embodiments of a connector, a delivery device, a container of flowable cement, a push rod for delivering a distal anchor, and a delivery rod for delivering a proximal anchor.

FIG. 16 shows the first anchorable member being further expanded by a mechanical assist, the opposition rod remaining engaged at the distal portion of the first expandable member, and being pulled proximally by the rod, which is still engaged at the distal end of the first anchorable member. This is an optional step in the method.

FIG. 17 shows an Allen wrench connector deployer extending through the second anchorable member to engage the connector and beginning to rotate the connector with respect to the two anchorable members in order to draw the two anchorable members together.

### DETAILED DESCRIPTION OF THE INVENTION

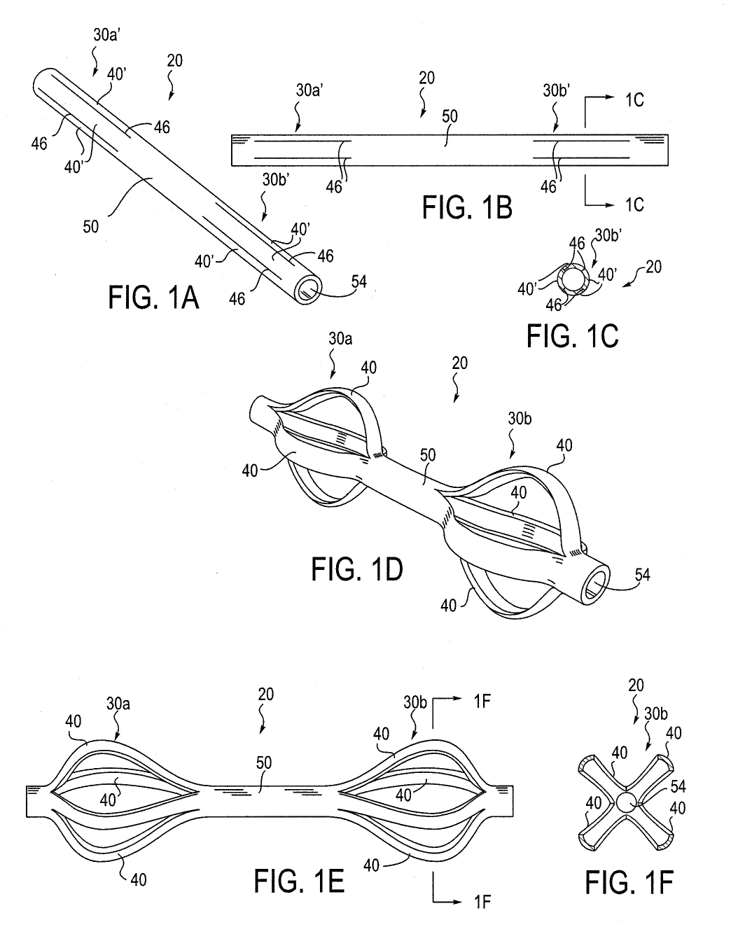

Described herein are transdiscal intervertebral body fusion systems and devices, and methods of using them to fuse compromised or damaged adjacent vertebrae. FIGS. 1-17 illustrate various embodiments of the system. Although the description specifies the use of embodiments of the transdiscal intervertebral body fusion system to fuse compromised or damaged adjacent vertebrae, the devices, systems and methods described herein may be used to fuse bones or regions of bone at sites of bone fracture, as described in U.S. patent application Ser. No. 12/041,607 of Chirico et al., as filed on Mar. 3, 2008, which is hereby incorporated by this reference. Sizes and specifics of device conformation and configuration are readily varied, and devices may be assembled so as to fit the specifics of a vertebral implant site. Further, the devices may be applied to regions of bone that include cancelous bone, cortical bone, or both types of bone.

In general, the transdiscal intervertebral body fusion devices included in the systems described herein include two anchorable (or anchoring) members connectable to or connected by a connector piece. These anchorable members typically include expanding (e.g., self-expanding) structures such as struts. As will be seen, struts may be highly variable in form, and may include for example, outwardly expanding structures the lead with flat, rounded, or sharp cutting edges. In some vertebral body sites, a cutting edge may be preferred as a way to cut into the bone most effectively to form an anchor, and in other sites, it may be preferred to lead with a flat of rounded surface that can provide more substantial outward support to a bone when the device is in its final anchoring position. Various embodiments and features of the devices, system and method will be described first with general references to FIGS. 1-20, and the embodiments represented therein will be detailed individually in greater detail thereafter.

A system for transdiscal intervertebral body fusion 20 may include two anchorable members 30 with an intervening connector piece 50. Anchorable members 30 can also be referred to as a first member 30a and second member 30b. In terms of the description herein, the first member 30a is implanted in a first vertebral body and the second member 30b is implanted in a second vertebral body. This terminology is neutral with respect to the relative cephalad or caudal position of the first and second vertebral bodies. In some embodiments of a method, the first member 30a is distal with respect to the second or thus proximal member 30b, distal referring to a position furthest from the delivery device or from the perspective of a delivery (or deployment) device that positions the device within adjacent vertebrae. The anchorable members typically have two configurations; one configuration is substantially unexpanded or collapsed, and may be substantially linear in form and orientation. This is the non-anchoring (or delivery) configuration of the member in which it may be deployed and positioned in a vertebral body site. The second configuration is an anchoring (or expanded) configuration, which typically includes a radially expanded structure. An anchorable member in a constrained or non-expanded configuration may be labeled as member 30′ (30 prime).

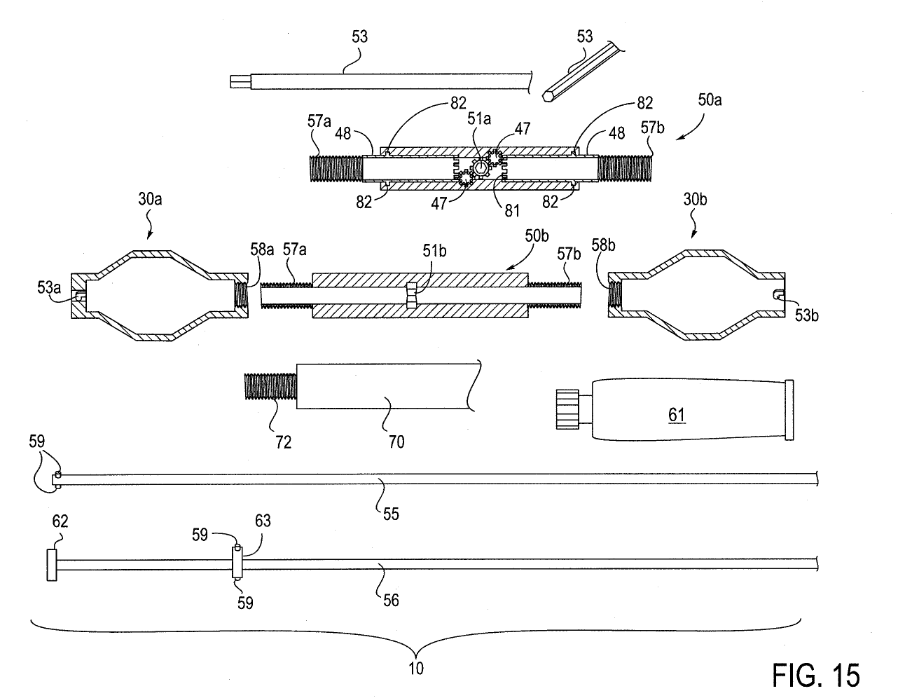

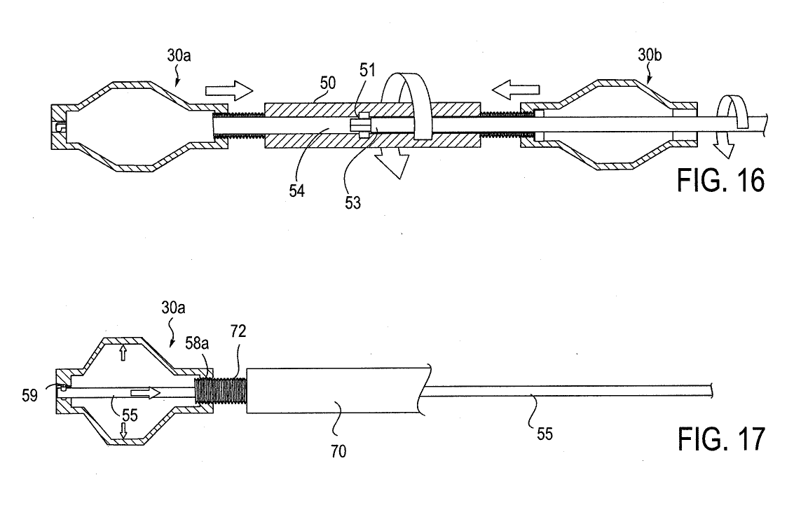

An assembled transdiscal intervertebral body fusion device may be formed in various ways. In some embodiments of device 20, two anchorable members 30 and a connector piece 50 are fabricated as a single integrated unit. In other embodiments, a second anchorable member 30b and a connector 50 are conjoined into a single integrated unit, and a first anchorable member 30a is a separate piece that is joinable with the integrated second anchor 30b and connector. In other embodiments, a first anchorable member 30a and a connector 50 are conjoined into a single integrated unit, and a second anchorable member 30b is a separate piece that is joinable with the integrated first anchor and connector. In some variations, a connector is a connector region extending from both anchorable members. In still other embodiments, a first or distal anchorable member 30a, a connector 50, and a second or proximal anchorable member 30b are each separate pieces that are conjoinable. In some embodiments of the transdiscal intervertebral body fusion device, the invention includes a kit of parts that may be assembled into a complete device 20 before implantation in a vertebral body site, or such parts may not be fully assembled until the time when they are being positioned within the vertebral body site. See FIG. 15 for an embodiment of a kit of parts. See FIGS. 16A-16O of U.S. patent application Ser. No. 12/040,607 of Chirico et al., filed on Mar. 3, 2008 (incorporated by this reference) for illustrations of one variation of a method of inserting a first anchorable member, a connector, and a second anchorable member in order to assemble a complete device.

In general, when any of the connector and both anchorable members are separate or separable, they may be connected in any appropriate manner. For example, they may be threaded (e.g., connected by screwing), or may be slidably connected (e.g., one or more anchorable members may slide over the connector region) that can interlock.

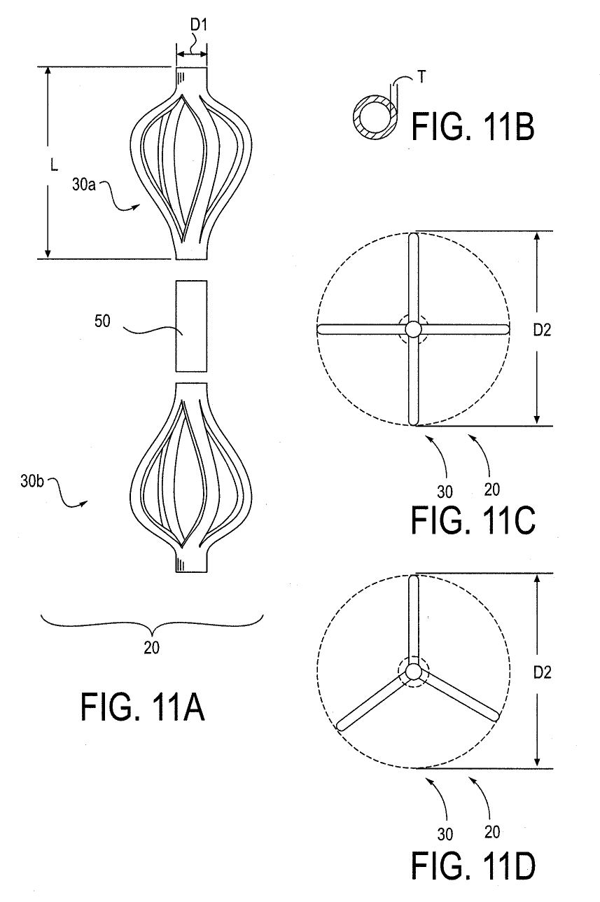

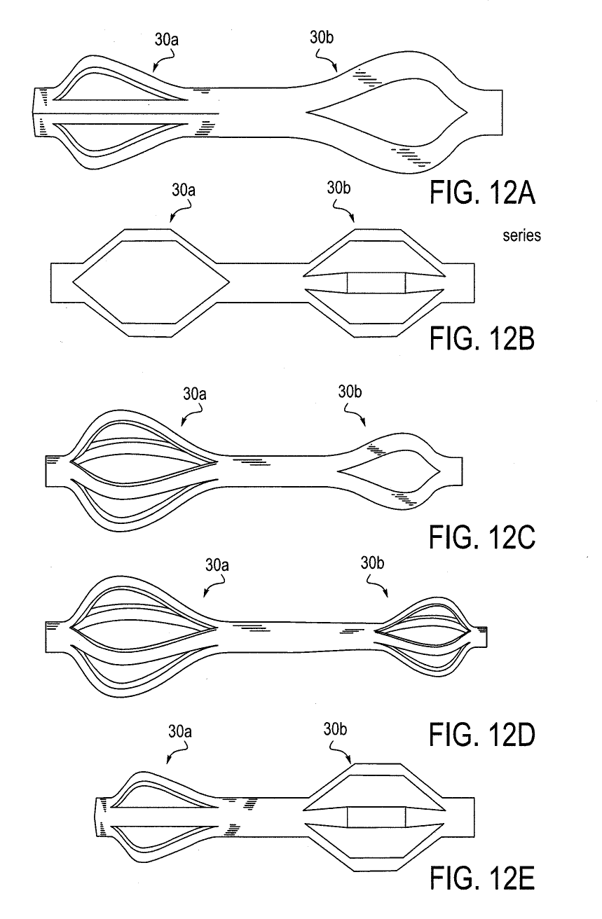

The dimensions of anchorable members 30 of a transdiscal intervertebral body fusion device 20 may be selected according to their intended site of use. The exemplary dimensions provided further below are to help in providing an understanding, and are not intended to be limiting. FIGS. 11A-11D show an embodiment of the device 20 and provides visual reference for various dimensions, and is described in further detail below. As noted above, the transdiscal intervertebral body fusion device 20 may be embodied as a kit of parts. These parts may have a modular character in that, in spite variations in size and form of some regions, there may be limited variation in some dimensions. For example, the diameter of the body may have a limited number of sizes so that parts are readily conjoinable around common features, particularly points of engagement or connection, such as threadable connections, as between a connector and anchorable members, and as in the size of the lumen extending through a connector and as such lumen or rod may further extend through anchorable members. A device 20 assembled from various parts could have identical first and second anchorable members, or the members could be dissimilar. The great variety of devices that may be generated from such a system allows for custom fitting of a device to the dimensions of a vertebral body and the surrounding locale; a few such exemplary devices with dissimilar first and second anchorable members are depicted in FIGS. 12A-12E.

Anchorable members 30 (and connector 50) may be formed from any appropriate and biocompatible material, such as and in particular, shape-memory materials. Anchorable members may be formed by “prebiasing” them into a shape such as an expanded (anchoring) shape. In some variations, components of the transdiscal intervertebral body fusion device are formed at least partially from a resiliently deformable material such as a plastic, metal, or metal alloy, stainless steel, for example, or a shape memory (and super-elastic) metal alloy such as Nitinol. A detailed description of materials that may be suitable for the fabrication of the present transdiscal intervertebral body fusion device may be found in U.S. patent application Ser. No. 11/468,759 (now U.S. Patent Application Publication 2007/0067034A1, published Mar. 22, 2007) which is incorporated by this reference in its entirety. In typical embodiments of an anchorable member, the biased or preferred state of the member is that of the radially-expanded anchoring configuration. In these embodiments, the unexpanded configuration that is appropriate for deployment and initial positioning within a vertebral body site is a constrained configuration, which is held in place either by radial or linear constraints.

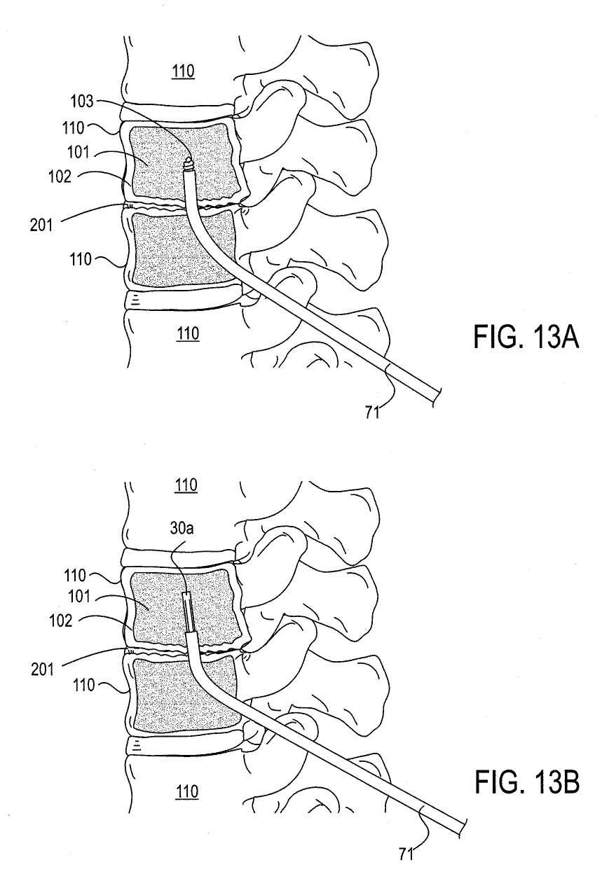

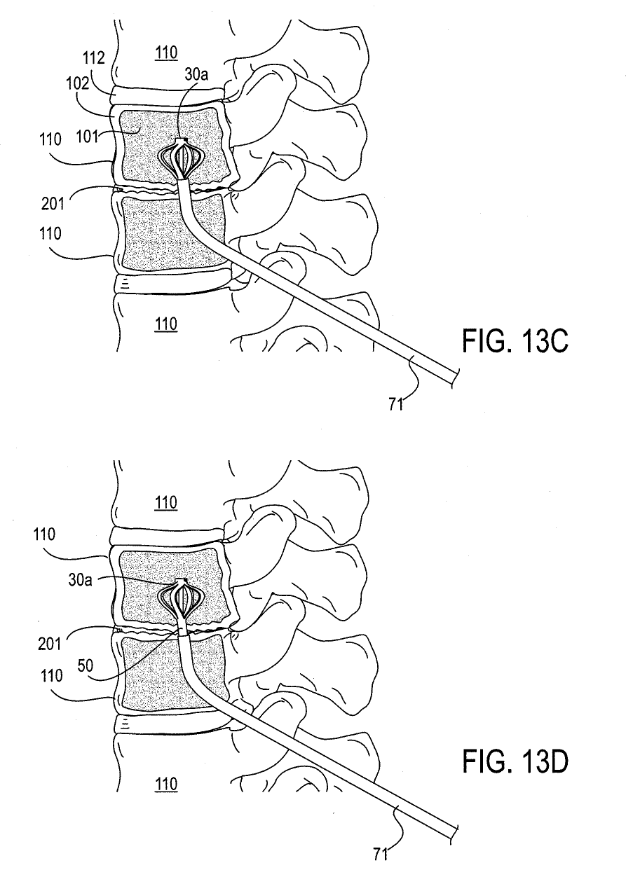

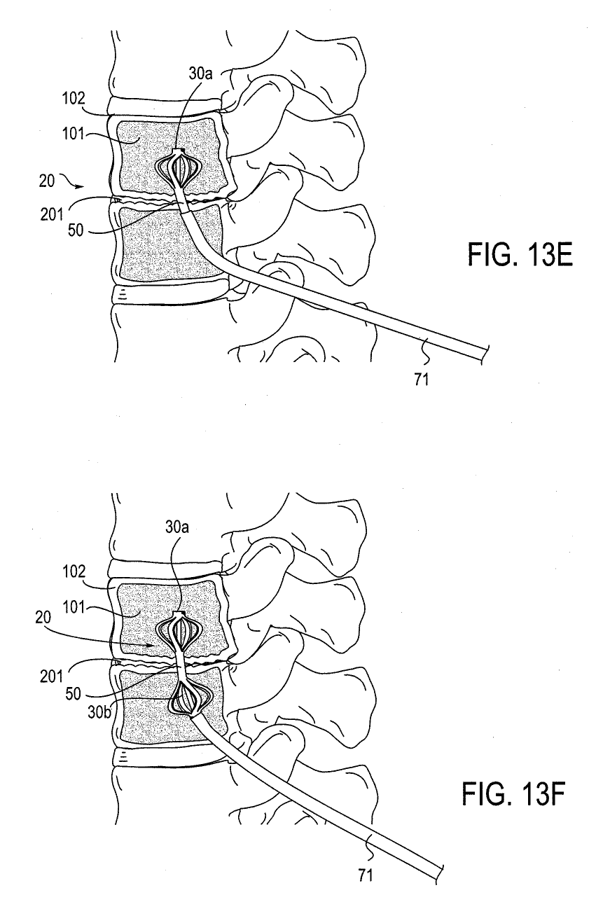

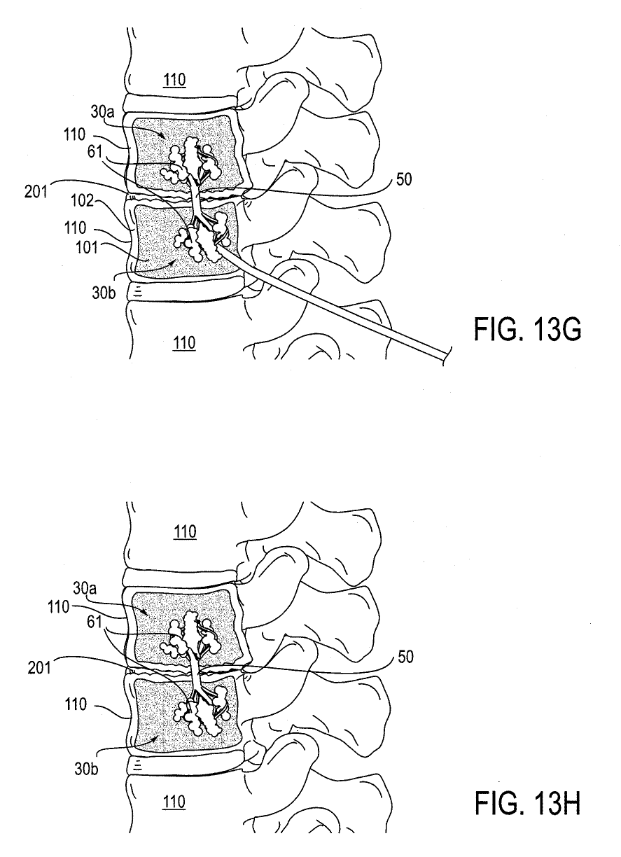

Embodiments of the invention may constrain an anchorable member 30′ in at least two ways, which will be described in greater detail below. Briefly, one approach is that of confining the member within an enclosing cannula or sleeve 71 that directly prevents radial expansion. A delivery device including a cannula or sleeve is shown in FIGS. 13A-13G, wherein a transdiscal intervertebral body fusion device configured as a single conjoined unit prior to delivery is implanted into adjacent vertebrae. In these embodiments, the delivery device may include a push rod, to distally eject a transdiscal intervertebral body fusion device. In some variations, the device, or regions of the device (e.g., the anchorable members) are placed under tension by the delivery device to prevent them from expanding. Radial expansion may shorten or contract the anchorable members of device. Thus, a delivery device may include one or more attachment sites to constrain the anchorable members from expanding. For example, a delivery device may apply tension to the anchorable members through a rod (e.g., a length-constrainment rod) extending distally from a delivery or deployment device 70. The rod may prevent shortening of length and radial expansion of anchorable members. The rod may be slidable within the delivery device, but can be held (e.g., locked) in an extended position to prevent deployment of the anchorable member. An example of a delivery device including a rod for applying or maintain tension is depicted in FIG. 15 and in FIGS. 16A-16O of U.S. patent application Ser. No. 12/040,607 of Chirico et al., filed on Mar. 3, 2008 (which is incorporated by this reference).

An anchorable device 20 may include two anchorable members 30 and a connector 50, and each of these components includes a passageway or channel 54 there through, which forms a continuous passageway 54 though the transdiscal intervertebral body fusion device. The passageway 54 may form a lumen through which a rod 57 may be inserted, and through which a flowable cementing or bone-filling material 61 may be conveyed. The passageway may also be a hollow tube 54 that can form a strengthening structural element for the device 20 as a whole. In some embodiments, only the connector portion includes hollow tube 54; in other embodiments, the hollow tube is included as a structural feature through the center of one or more of the anchorable members (see FIG. 8, for example). The connector and/or tube 54 also may also be configured so that the anchorable members 30 may be moved closer or further apart from each other. For example, the connector and/or tube may be threaded such that a turnbuckle-style rotation of either the connector or one or more of the anchorable members may draw the members closer together, as shown in FIG. 17.

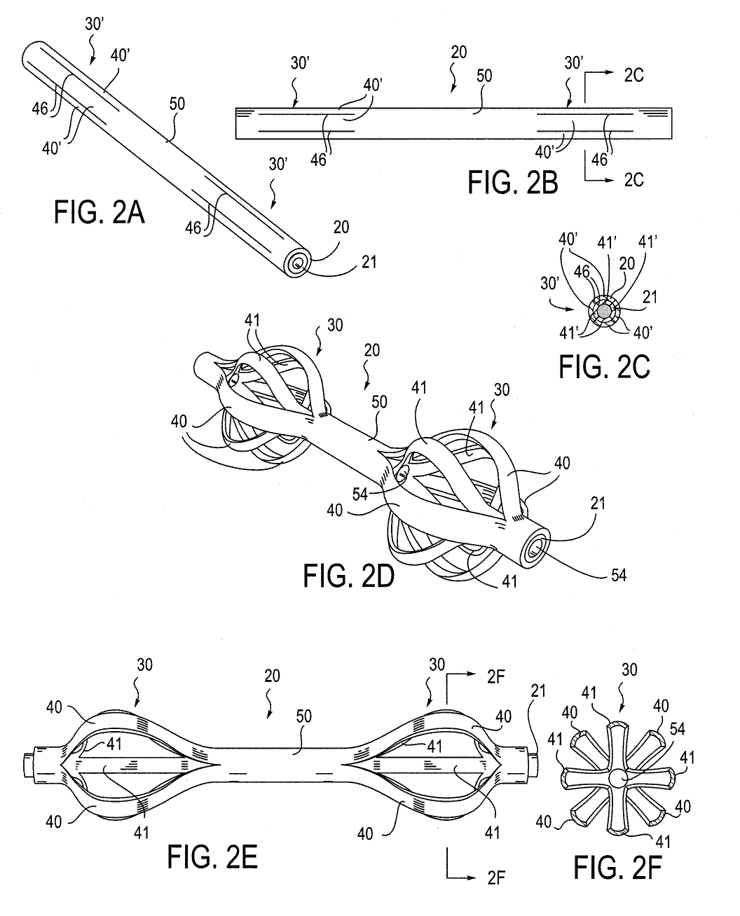

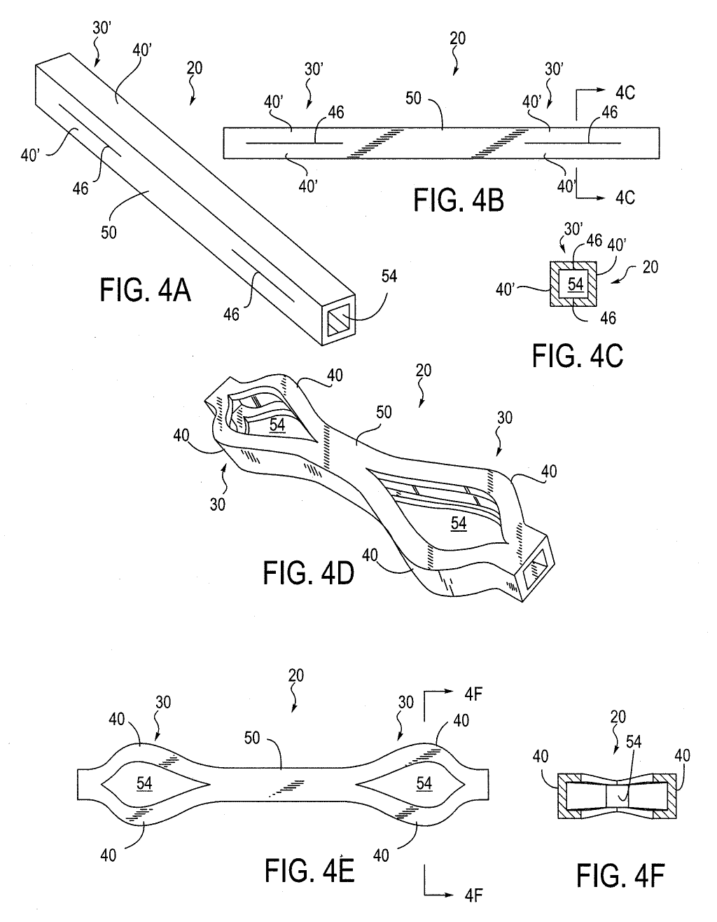

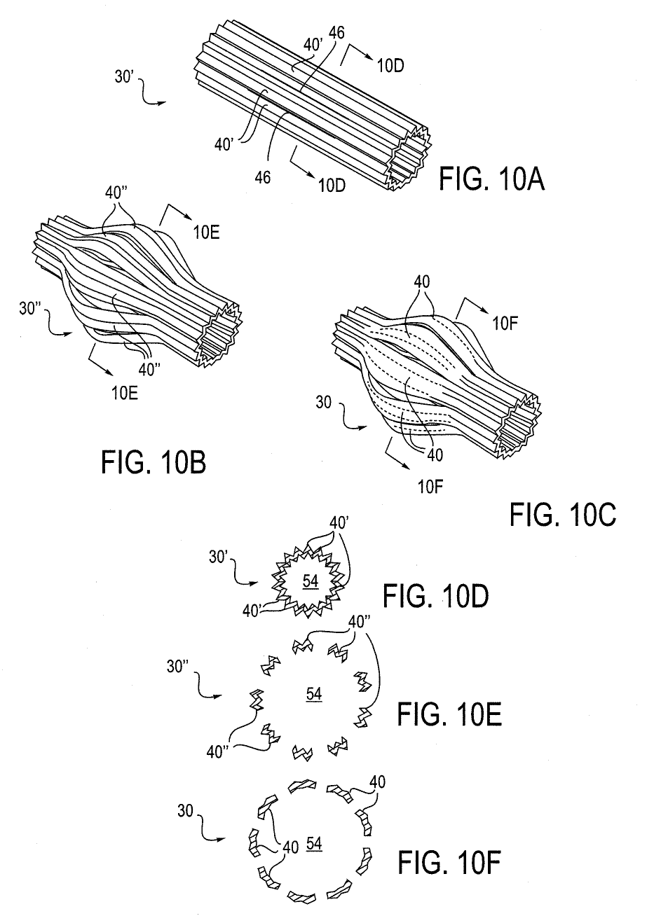

In its constrained (delivery- or deliverable) configuration, an anchorable member 30′ may be in the form of a substantially hollow tube. In some variations, the cross-section of the transdiscal intervertebral body fusion device is substantially circular or oval (as in FIGS. 1 and 2). In some variations, it is a sided-structure, e.g., having three sides, four sides, or more than four sides (as in FIGS. 4, 5, and 9). In an embodiment with four sides, a rectangular configuration may have four sides of equal length. Further variations of the cross sectional profile may occur in other embodiments. For example, the vertices or corners of a sided-embodiment may be pinched or crimped in (FIGS. 9A and 9B), this configuration may create a more acute cutting edge on the struts as they undergo their self-expansion upon release of the device from constraint. In other embodiments, the surface may be substantially round in profile, but embellished with linear corrugation, as show in FIGS. 10A-10C. In this configuration, the linear folds of the struts may impart strength to the struts that remains even in the expanded configuration of the struts.

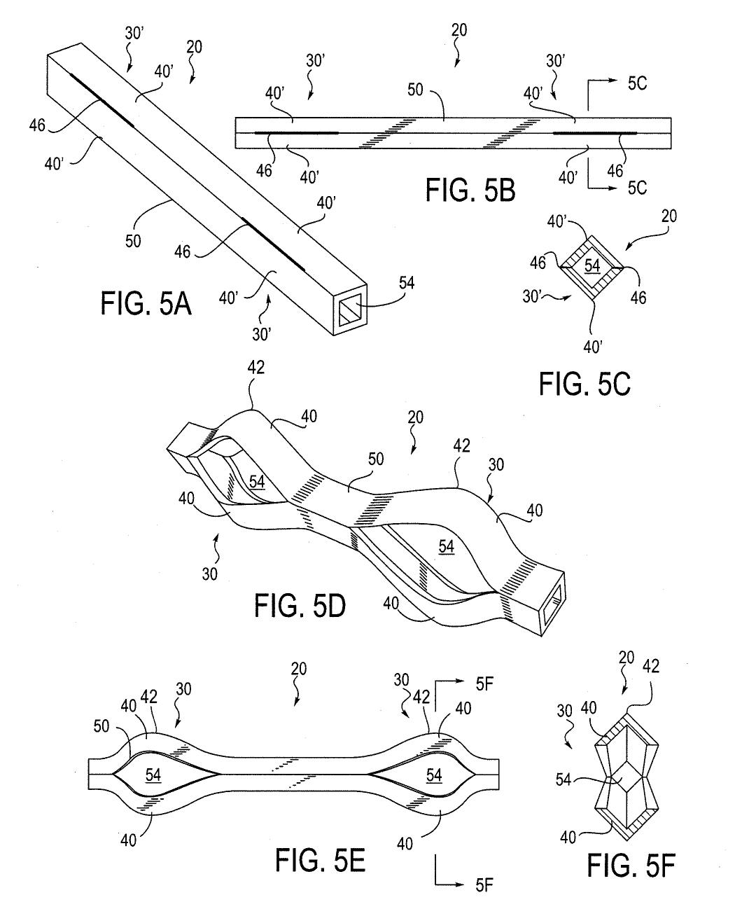

As described in U.S. patent application Ser. No. 11/468,759 (Pub No. US 2007/0067034 A1) and U.S. Provisional Patent Application No. 60/916,731, slots or slits 46 may be cut lengthwise in a tube to form nascent struts 40. With metallurgical methods well known in the art such as heat treatment, the struts 46 may be configured into a preferred configuration such as a bow. In some device embodiments, the configuration of bowed struts may be linearly symmetrical or substantially symmetrical (as shown in FIGS. 5, 9, and 10), and in other embodiments, the bow may be asymmetrical (as shown in FIGS. 1-4), with the maximal expanded portion skewed either toward the distal or proximal end of an anchorable member. Other configurations of symmetrical and asymmetrical struts may also be used.

An anchorable member 30 having three struts (FIG. 7, for example) comprising the body 45 of device 20 typically has a triangular cross section, the struts formed by slots cut through the surface of each of the three sides. In an embodiment where the triangle of the cross-section is equilateral, the struts are radially distributed equally from each other, with 120 degrees separating them (see FIG. 11D). In other embodiments, where the triangle of the cross sections is not an equilateral triangle, the radial angles of struts may include two that are equal, and a third angle that is not equal to the other two. There may be some benefits associated with anchorable member embodiments with three struts compared with four or more struts. The struts (of a three-strut anchorable member) formed are wider, and thereby stronger than those of anchorable members having four struts emanating from a device body of the same diameter.

In some four-strut variations, the body 45 of the device 20 may be either square or circular in cross section, and the four struts 40 emanating from the body are typically equally spaced apart at 90 degrees, or they may be radially distributed such that the angles formed include two angles greater than 90 degrees and two angles less than 90 degrees. A body 45 with a square cross section typically is appropriate to support struts that are spaced apart by 90 degrees, the strut-forming slots positioned centrally lengthwise along the body (FIGS. 4A-4F). This configuration also imparts a 90 degree leading edge on struts 40 formed therefrom, such an edge being useful in cutting through bone. In many embodiments of the invention, efficiency in cutting through bone, either or both cortical bone or cancellous bone, is advantageous. Cutting may separate bone mass to allow strut movement through bone with minimal compression of bone, and thus minimal disturbance of bone tissue in regions adjacent to the path of separation. Bone (particularly cortical bone) may be cut only slightly, and may serve to help anchor the device in or to the bone. In other embodiments, it may be desirable that struts 40 have a surface that presents a flat face for vertebral body support, e.g., expandable members having a circular cross section (as illustrated in FIGS. 1A-1F and 2A-2F).

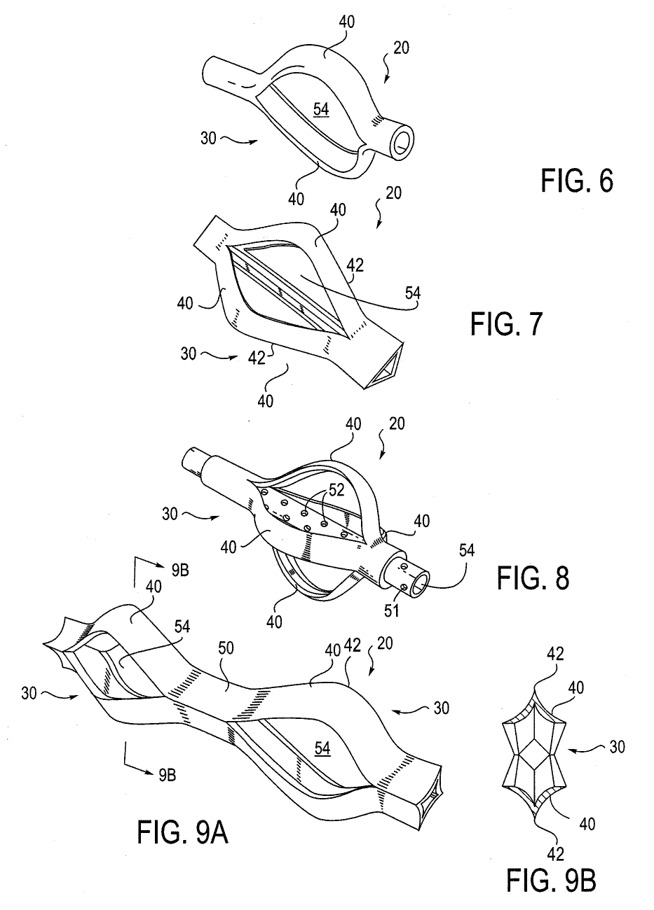

In some variations, the anchorable member includes only two struts. In these variations, the 45 of a device 30 may be circular (FIG. 6) or square (FIGS. 4A-4F) in cross section. In embodiments having a square cross section, lengthwise slots 46 may be made at opposite vertices of the square, in which case the two struts formed therefrom have a 90 degree leading edge (FIGS. 5A-5F). For example, a body having a circular cross section may include lengthwise slots 46 that may be made at radially opposite positions, in which case the two struts formed therefrom have a broad leading edge (FIG. 6). In some embodiments of a transdiscal intervertebral body fusion device 20, a broad leading edge may be beneficial if the leading edge is intended to provide support to a vertebral body surface from within.

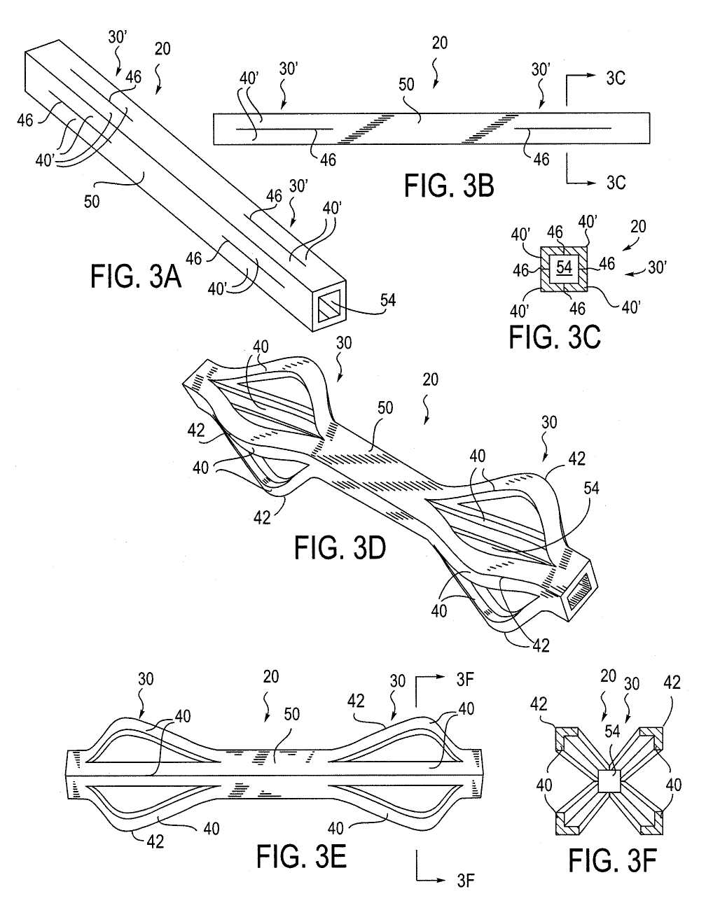

As mentioned, the struts 40 may be formed by cuts or slots 46 in the body of the device 20 and may include a sharp cutting edge 42 useful for cutting, scoring or securing to bone (either cancellous bone 101 or cortical bone 102) as the struts radially expand upon being released from constraint (FIGS. 3A-3F, 5A-5F, and 9A-9B). A sharp edge may be derived from a vertex or corner of the device body as seen in cross section. Thus, for example, a rectangular body or a triangular body can generate struts with a sharp leading edge as the struts expand. In typical embodiments, for example, where struts are formed from the body of an anchorable device with a rectangular cross section, cuts in the metal to create slots are made in the central portion of sides of the rectangle, and struts 40 are formed at the vertices of the rectangle. Thus, in some embodiments, the cutting edge 42 of a strut 40 may have a leading angle of about 90 degrees. In other embodiments of an anchorable member 30 with a rectangular cross sectional profile, the vertices of the rectangle may be crimped or pinched in order to create corner angles that are more acute than 90 degrees (FIGS. 9A and 9B). In embodiments such as these, the cutting edge 42 or a strut 40 may have a leading angle more acute than 90 degrees. In embodiments of an anchorable member 30 with an (equilateral) triangular cross section, the vertices of the triangle have an angle of 60 degrees, and thus struts 40 formed from such vertices have a cutting edge 42 with an angle of 60 degrees.

In some embodiments of a transdiscal intervertebral body fusion device 20, the first anchorable member 30a and the second anchorable member 30b are identical (e.g., FIGS. 1-6). In other embodiments of a transdiscal intervertebral body fusion device 20, the first 30a and second 30b anchorable members are dissimilar (FIGS. 12A-12D). A transdiscal intervertebral body fusion device may include anchorable members that are different in size (e.g., length of body 45, length of struts 40, differences in diameter of the body 45), different in the radial expansiveness of the released configuration of struts 40, different with regard to the symmetry or asymmetry of bowed struts 40, or different in any other anchorable member parameter. By such variations in form of the two anchorable members 30, a transdiscal intervertebral body fusion device 20 may be tailored to suit the particular dimensions of a target vertebral body site. As described above, a device 20 may be further tailored or fitted to a target vertebral body site by any of the variations in size provided by embodiments of anchorable members 30 and their components, such as struts 40 or connector 50.

Some embodiments of a transdiscal intervertebral body fusion device 20 may have a double-body, including an internal anchorable member within an external anchorable member (FIGS. 2A-2F). The benefit provided by this general configuration is that it provides more surface area (e.g., twice as much) for anchoring within a given anchoring volume of bone than does a single anchoring member. Typically, the number of struts in the companion internal and external bodies are the same, and are radially staggered with respect to each other, so that the struts of the inner body may emerge in the spaces between the struts of the outer body. The struts of the inner and outer bodies may be of about the same length and bowed outwardly to about the same degree, as they are in FIGS. 2A-2F. In other embodiments, the struts of the inner body may be shorter in length, or bowed outward to a lesser degree than the struts of the outer body.

A transdiscal intervertebral body fusion system 10 may include various delivery devices, two of which will be described. By way of example, a delivery device may be a sleeve or cannula 71 which directly constrains the radial expansion of embodiments of device 20 for deployment (FIGS. 13A-13H). Deployment occurs by means of a push rod extending distally in the delivery device to a point of contact on the proximal surface of the second or proximal anchorable member 30b. By pushing the device 20 distally and at the same time withdrawing the cannula from an implantation site, the first or distal anchorable member 30a emerges from the cannula and self-expands as it is released from the lateral or circumferential constraints of the cannula. As the cannula is withdrawn further in the proximal direction from an implantation site and simultaneously continuing to push the device distally out of the cannula, a connector portion 50 and a second or proximal anchorable member 30a emerge in sequence. As the second anchorable member is released from the circumferential constraints of the cannula it self-expands, as did the first anchorable member. This sequence concludes the initial stage of positioning and implantation, which then may be followed by adjustments that include a mechanical assist to further expansion of the anchorable members (FIG. 16) or bringing the anchorable members closer together (FIG. 17.) In a variation of the method, pieces of a transdiscal intervertebral fusion device may be delivered to an implant site individually and assembled in place. An advantage offered by the delivery of device pieces and assembling in place (rather than delivering an integral or already assembled device) is that smaller pieces (single anchorable members, a connector, or a conjoined anchorable member and connector) can negotiate tighter delivery paths and more acute channel angles than can a fully assembled or integrally-formed device.

A second exemplary delivery device 70 illustrated herein generally constrains the transdiscal intervertebral body fusion device to a linear configuration and prevents expansion of struts by applying tension across at least a portion of the device to prevent contraction of shortening of the body of the device. Thus the direct constraint holding the anchorable member in a delivery configuration is one that prevents linear contraction of the anchorable member portion of the device; however the constraint consequent to the linear constraint is a prevention of radial expansion that accompanies length contraction or reduction. Embodiments of this delivery device may be similar to embodiments of delivery devices disclosed in detail in U.S. Provisional Patent Application No. 60/906,731, filed on May 8, 2007, and which is hereby incorporated in its entirety. An example of this method of device delivery is shown in FIGS. 16A-16O of U.S. patent application Ser. No. 12/040,607 of Chirico et al., filed on Mar. 3, 2008 (which is incorporated by this reference). That series of figures shows the implantation of a device across a fracture region in a matter that is analogous to intervertebral body site shown in FIGS. 13A-13F. By this approach a first anchorable member is delivered and positioned in a first vertebral body, a connector is then delivered to the portion of the channel prepared for the device that spans the intervertebral space and through the intervening disc, and is connected to the proximal end of the first anchorable member. Finally, a second anchorable member is delivered to channel site within the second vertebral body and connected to the proximal end of the connector, to complete the assembly of the device.

A transdiscal intervertebral body fusion device may be delivered by providing a delivery device that constrains the anchorable members from contraction, as just described and as depicted in FIGS. 13A-13F. The delivery device can be used to sequentially expand a first anchorable member, and a second anchorable member, either sequentially or simultaneously. The device may be inserted with all of the components of the transdiscal intervertebral body fusion device attached (e.g., fully assembled) or with them in components that are joined after (or during) delivery.

As described above, some embodiments of device 20 may be fabricated from a superelastic shape memory alloy such as Nitinol, in which case struts 40 may be configured to self-expanding when released from constraint in a radially non-expanded (or linear form). When implanted in bone, particularly in hard cortical bone, expansion of struts may be resisted by surrounding bone. Facing such resistance, expandable struts 40 may not expand to their full potential. Inasmuch as greater anchoring stability is associated with full radial expansion, it may be advantageous to mechanically assist struts in their expansion. Additional mechanical expansion may be achieved by drawing the distal and proximal ends of anchorable members closer together. FIG. 15 shows an exemplary mechanism by which mechanical force is applied to partially expanded struts in order to assist in their full expansion.

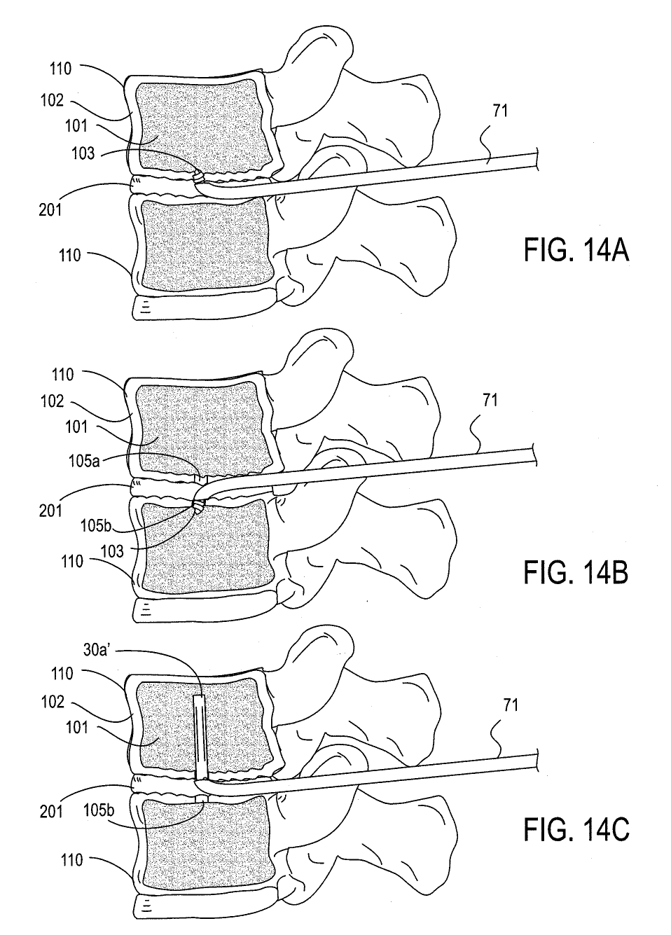

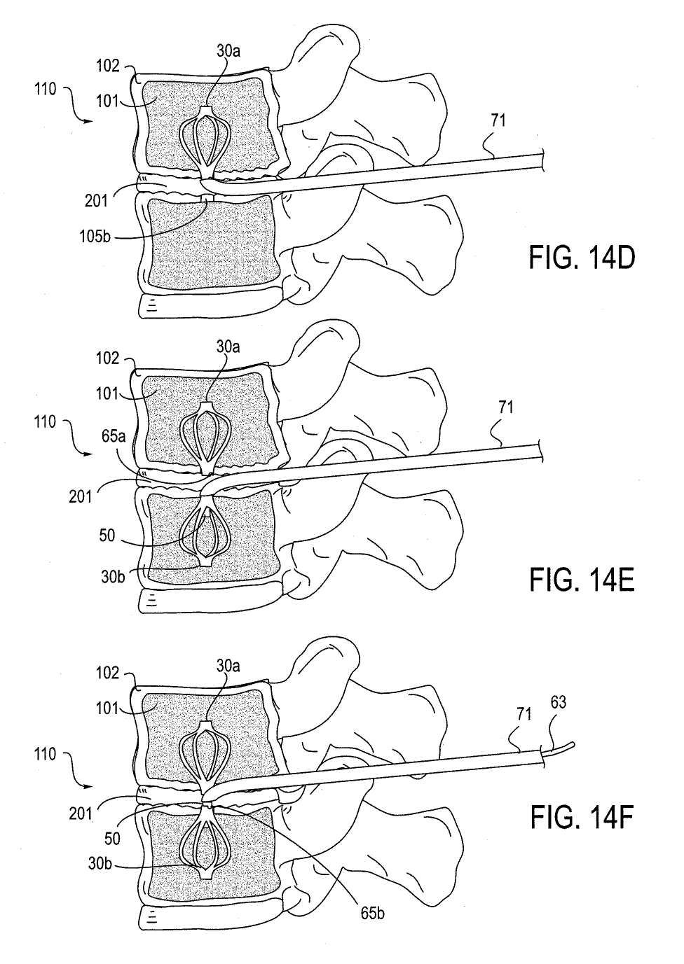

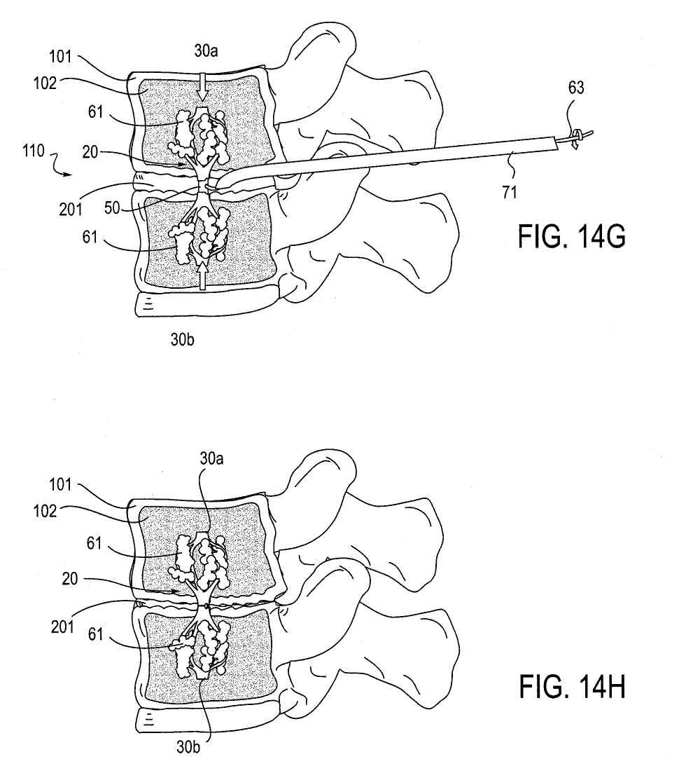

There are a number of routes by which to insert a transdiscal intervertebral body fusion device into two adjacent vertebrae. In one approach, for example and as described in detail below and as depicted in FIGS. 13A-13H, a channel for the device is created by percutaneously entering a side of one vertebral body that proceeds through an end plate of the body, into the intervertebral space, traversing the intervertebral disc, entering the end plate of the adjacent vertebral body and terminating within its interior. In another exemplary approach, as described in detail below and as depicted in FIGS. 14A-14H, an access channel is opened in the intervertebral space and penetrating the disk as necessary. From that access channel, a cephalad channel is opened into the cephalad vertebral body and a caudal channel is opened into the caudal vertebral body. The cephalad and caudal channels, aligned at their base, then form a single continuous channel into which the transdical intervertebral body may be implanted. The approach through the side of a vertebral body (FIGS. 13A-13H) offers the advantage of a relatively straightforward implantation path. The approach through the intertebral space (FIGS. 14A-14H) provides the advantage of sparing the one vertebral body the injury associated with providing the entry channel.

Following implantation of a transdiscal intervertebral body fusion device, a flowable bone filling composition or cement 61 such as PMMA (polymethylmethacrylate) may injected into the spinal region through a trocar and cannula system into the passageway 54 of a device 20. There are many suitable materials known in the art for filling in vacant spaces in bone, some of these materials or compositions are biological in origin and some are synthetic, as described in U.S. patent application Ser. No. 11/468,759, which is incorporated by reference herein. From the passageway, the material flows into the open space within the anchorable members and to some degree, into the peripheral area surrounding the device. The flowable cementing material may contain radiopaque material so that when injected under live fluoroscopy, cement localization and leakage can be observed.

Another example of bone cementing material is provided by a ceramic composition including calcium sulfate calcium hydroxyapatite, such as Cerament™, as manufactured by BoneSupport AB (Lund, Sweden). Ceramic compositions provide a dynamic space for bone ingrowth in that over time the compositions may resorb or partially resorb, and as a consequence progressively provide new space for ingrowth of new bone. Bioactive agents may also be included in a cementing composition, such as osteogenic or osteoinductive peptides, as well as hormones such at parathyroid hormone (PTH). Bone Morphogenetic Proteins (BMPs) are a prominent example of effective osteoinductive agents, and accordingly, a protein such as recombinant human BMP-2 (rhBMP-2) may included in an injected bone-filling composition. In this particular context, BMPs promote growth of new bone into the regions in the interior of the expanded struts and around the periphery of device 20 in general, to stabilize the device within new bone. A more fundamental benefit provided by the new bone growth, aside from the anchoring of the device 20, is simply the development of new bone which itself promotes healing of a transdiscal intervertebral body fusion. In some embodiments of the invention, antibiotics may be included, particularly when there is reason to believe that the vertebral site may have been infected. With the inclusion of bioactive agents such as bone growth or differentiation factors, or antibiotics or other anti-infective agents, embodiments of the transdiscal intervertebral body fusion device become more than a fusion or fixation device, as such embodiments take on the role of an active therapeutic or drug delivery device. In general, any appropriate flowable material may be injected into the passageway formed through the transdiscal intervertebral body fusion device. In some variations the device (e.g., the proximal end of the transdiscal intervertebral body fusion device) may be adapted to receive a device for delivering flowable material.

Examples of transdiscal intervertebral body fusion devices, system and methods of using them are provided below, including methods of implanting the device into adjacent vertebrae to stabilize the vertebrae, as particularly detailed in FIGS. 1-20.

For example, FIGS. 1A-1F provide views of a transdiscal intervertebral body fusion 20 with a circular body having a lumen 54 and two anchorable members 30a, 30b, each with four radially expandable struts 40′, the struts having a flat expanding surface, and a connector portion 50. FIG. 1A is a perspective view of the body of the device. FIG. 1B is a side view of the body of the device showing slots 46 to be cut from which struts will emerge. FIG. 1C is a cross-sectional view of the device. FIG. 1D is a perspective view of the device after the struts 40 have radially expanded. FIG. 1E is a side view of the device after the struts have radially expanded. FIG. 1F is an end view of the device after the struts have radially expanded. A number of structural features of embodiments of the dual-anchoring system 20 described herein, such as slots 46, struts 40, and anchorable members in general, as well as methods of delivery and implantation are similar to features of a vertebral body stabilization device with a single anchorable member, as described in U.S. patent application Ser. No. 11/468,759, which is incorporated into this application, and which may help in the understanding of the present invention.

FIGS. 2A-2F provide views of an internal-external, or double-bodied, transdiscal intervertebral body fusion device, the outer body 20 surrounding an internal body 21. Each body has a lumen 54 and two anchorable members 30, each with four expandable struts 40′, the struts 41 of the internal body and the struts 40 of the external body staggered with respect to each other, and a connector portion 50. FIG. 2A is a perspective view of the body of the device. FIG. 2B is a side view of the body of the device showing slots 46 to be cut from which struts will emerge. FIG. 2C is a cross-sectional view of the device. FIG. 2D is a perspective view of the device after the struts 40 have radially expanded. FIG. 2E is a side view of the device after the struts have radially expanded. FIG. 2F is a cross-sectional view through the struts of the device after the struts have radially expanded.

FIGS. 3A-3F provide views of a transdiscal intervertebral body fusion device 20 with a rectangular body having a lumen 54 and two anchorable members 30, each with four radially expandable struts 40′, each emanating from a slot 46 cut through a flat surface of the body and expanding with a leading sharp edge 42, and a connector portion 50. FIG. 3A is a perspective view of the body of the device. FIG. 3B is a side view of the body of the device showing slots 46 to be cut from which struts will emerge. FIG. 3C is a cross-sectional view of the device. FIG. 3D is a perspective view of the device after the struts 40 have radially expanded. FIG. 3E is a side view of the device after the struts have radially expanded. FIG. 3F is a cross-sectional view through the struts of the device after the struts have radially expanded.

FIGS. 4A-4F provide views of a transdiscal intervertebral body fusion device 20 with a rectangular body having a lumen 54 and two anchorable members 30, each with two radially expandable struts 40′ emanating from length-wise cuts in a flat surface of the body and expanding with a leading flat edge, and a connector portion 50. FIG. 4A is a perspective view of the body of the device. FIG. 4B is a side view of the body of the device showing slots 46 to be cut from which struts will emerge. FIG. 4C is a cross-sectional view of the device. FIG. 4D is a perspective view of the device after the struts 40 have radially expanded. FIG. 4E is a side view of the device after the struts have radially expanded. FIG. 4F is a cross-sectional view through the struts of the device after the struts have radially expanded.

FIGS. 5A-5F provide views of a transdiscal intervertebral body fusion device 20 with a rectangular body having a lumen 54 and two anchorable members 30, each with two radially expandable struts 40′ emanating from length-wise cuts at a vertex of the rectangle, each strut expanding with a leading sharp edge 42, and a connector portion 50. FIG. 5A is a perspective view of the body of the device. FIG. 5B is a side view of the body of the device showing slots 46 to be cut from which struts will emerge. FIG. 5C is a cross-sectional view of the device. FIG. 5D is a perspective view of the device after the struts have radially expanded. FIG. 5E is a side view of the device after the struts 40 have radially expanded. FIG. 5F is cross-sectional view of through the struts of the device after the struts have radially expanded. Device embodiments such as these depicted in FIG. 5, FIG. 4, and FIG. 9 with two radially expandable struts may be particularly advantageous for fixing fractures in a flat bone such as a skull plate (FIG. 14) or in any bone or fracture site that is small, or has a narrow planar constraint.

As mentioned above, although the examples shown in FIGS. 1A and 2A are transdiscal intervertebral body fusion devices that are integrally formed, the anchorable regions may be separate and attachable including separate and attachable to a connector) via the connector region. Further, any of embodiments described herein may include one or more attachment regions for attachment to a delivery device (including both distal and proximal attachment sites), and attachment to a length-adjusting device (for changing the spacing between the anchorable members), or attachment to a source of flowable material (e.g., cement). Attachment sites may be threaded attachment sites, interlocking attachment sites (e.g., keyed attachment sites), gripping attachment sites, or any appropriate releasable attachment site.

FIGS. 6-8 show exemplary anchorable members 30 which may be understood as components of a complete double-anchored device 20, these single anchorable members being presented to exemplify particular features comparative way. FIG. 6 provides a view of a single anchorable member 30 with two radially opposed struts 40 in an expanded configuration, the member being a component joinable with a connector portion and a second anchor to form a complete transdiscal intervertebral body fusion device. FIG. 7 provides a view of a single anchorable member 30 with three radially distributed struts 40 in an expanded configuration, the member being a component joinable with a connector portion and a second anchor to form a complete transdiscal intervertebral body fusion device.

FIG. 8 provides a view of a single anchorable member 30 with four radially opposed struts 40 in an expanded configuration, the member being a component joinable with a connector portion and a second anchor to form a complete transdiscal intervertebral body fusion device, the anchorable member further including a central rod or tube 54 that forms a continuous passageway with a connector in the fully assembled device. In some variations, the connector is the central tube 54 shown, and the anchorable members 30 may be slidable thereon. The anchorable members may be locked into position. In some variations, the connector does not lock to the anchorable members. The connector portion and/or the rod may include holes 52 from which a flowable bone cement may be ejected. Lumen 54 as seen in FIG. 8 in the form of a central rod extending through the anchorable member 30 may also be understood as to include the contiguous open space, in general, within the interior of expanded struts 40 as depicted in FIG. 6 and FIG. 7.

FIGS. 9A and 9B provide views of a transdiscal intervertebral body fusion device 20 with a rectangular body and two anchorable members 30, each with two radially expandable struts emanating from length-wise cuts at a vertex of the rectangle. This device is similar to that depicted in FIG. 5 except that the corners of the rectangle have been pinched or crimped in, giving the corner an internal angle more acute than 90 degrees. These acute corners become the leading and cutting edge 42 of a strut 40 as it expands, and in this embodiment the leading edge is particularly sharp. FIG. 9A is a perspective view of the body of the device. FIG. 9B is a view of one strut of the device after radial expansion.

FIGS. 10A-10F show a portion of one anchorable member of an embodiment of a double-anchored transdiscal intervertebral body fusion device with a linearly corrugated or crenellated surface, from which nine expandable struts 40′ emanate. FIG. 10A shows the anchorable anchorable member 30′ in a linearly constrained, non-radially expanded configuration. Slots 46 are present in the inner vertex of corrugations. FIG. 10B shows the anchorable member 30″ with expansion of the struts 40″ to a first position, which may either be a partial or fully self-expanded configuration, depending on the preferred configuration of the heat-treated shape memory metal. FIG. 10C shows expansion the anchorable member 30 and the expandable struts 40 to a second position, more expanded than the first position of FIG. 10B. FIG. 10D shows a radial cross sectional view of anchorable member 30′ at position 10D of FIG. 10A, showing the corrugated nature of the body of the anchorable member. FIG. 10E shows a radial cross sectional view of anchorable member 30″ at position 10E of FIG. 10B, showing the M-shaped cross-sectional profile the expanded or partially-expanded struts 40″. FIG. 1F shows a radial cross sectional view of anchorable member 30 at position 10F of FIG. 10C, showing the flattened M-shaped cross-sectional profile of fully expanded struts 40.

FIGS. 11A-11D show one example of a transdiscal intervertebral body fusion device that has been exploded into three parts, as well as cross sectional views of the body of the device, and of the anchorable members in their expanded configuration. This figure may illustrate the location of various dimensions of the device. Dimensions of anchorable members 30 of a transdiscal intervertebral body fusion device 20 may be chosen according to their intended site of use. The exemplary dimensions provided here are to help in providing an understanding of the invention, and are not intended to be limiting. For example, in some embodiments, the length L of the body 45 of an anchorable member when the struts 30 are in the radially expanded configuration may vary from about 7.5 mm to about 48 mm, and in particular embodiments, from about 24 mm to about 40 mm. In other embodiments, for particular applications, the length of the body may be less than 7.5 mm or greater than 48 mm. The thickness T (FIG. 11B) of the tube wall of a tubular body 45 may vary from about 0.2 mm to about 2.5 mm, and in typical embodiments is about 0.5 mm in thickness. The outside diameter D1 of the body of the device in its linear configuration may vary. In one variation, the outer diameter varies between about 1 mm to about 8 mm in diameter. FIG. 11D shows a cross sectional view of an alternative embodiment with three struts, radially distributed at 120 degrees, is included to convey the applicability of this diameter measurement even when struts do not form a straight-line diametric structure as can four struts. In the context of a released or anchoring configuration of an anchorable device 30, the struts 40 may expand to a maximal radial distance (FIGS. 11C and 11D) from about 3.5 mm to about 22 mm, to create a maximal diameter D2 (extrapolating the strut profiles to form a circle enclosing the maximal points of expansion) of about 7.5 mm to about 44 mm. In other embodiments, for particular application to particular vertebral sites, the maximal expansion diameter may be less than 4 mm or greater than 25 mm.

FIGS. 12A-12E show various embodiments of transdiscal intervertebral body fusion devices that have dissimilar first and second anchoring or anchorable members for custom fitting into adjacent vertebral bodies. FIG. 12A is a device with a three-strut anchorable member 30a and a two-strut anchorable member 30b, in each case that struts curvilinear and asymmetrically bowed. FIG. 12B is a device with a two-strut anchorable member 30a and a four-strut anchorable member 30b, the struts on each anchor are symmetrically bowed, have substantially straight segments, and are about the same size. FIG. 12C is a device with a four-strut anchorable member 30a that is significantly larger than its two-strut companion 30b. FIG. 12D is a device with two four-strut anchorable members, both asymmetrically bowed, one anchorable member 30a being larger than the other 30b. FIG. 12E is a device with one three-strut anchorable member 30a and a larger four-strut anchorable member 30b, the struts being symmetrical with substantially straight segments. These are but a few examples of what can be understood to be a very large range of combinations of anchorable members that can be joined together in order to fit the specific dimensions or conditions of compromised vertebrae.

Preliminary to forming a disc-traversing implant site within two adjacent vertebrae channel to receive a transdiscal intervertebral body fusion device 20, the vertebrae may be aligned in a natural or a desirable position. After a channel is prepared, a device is positioned within the channel and deployed. FIGS. 13A-13H provide views of the deployment of an integrated transdiscal intervertebral fusion device into two adjacent vertebral bodies 110 through a channel that enters the wall of one of the vertebral bodies, and continues through the disc 201 and terminates in the interior of the adjacent vertebral body, the device having an internal-external double body configuration, each body having four expandable struts. FIG. 13A shows a delivery device or cannula 71 being used to guide a drill 103 into through the side of caudal vertebral body 110, having penetrated through the disc 201 cephalad to it, and into cephalad vertebral body 110. By drilling this passageway, the drill has created a channel for the positioning and deployment of a fusion device 20, which is seen in a completely deployed from in FIG. 13F. It can further been seen that the drill 103 has penetrated cortical bone 101 that comprises the periphery and end plates of vertebral bodies 110, and the cancellous bone within the interior of the vertebral bodies.

Deployment of device 20 into the implant site is shown in FIGS. 13B-13F. FIG. 13B shows deployment of the first or distal anchorable member 30a, still in its constrained or linear configuration, because its proximal portion is still within cannula 71. Slots 46, seen in various embodiments of FIGS. 1A, 2A, 3A, and 4A can be seen in emerging member 30a (unlabeled). Also not seen in this figure is a push rod that extends through cannula 71, with which an operator pushes the device forward through the cannula, while at the same time, withdrawing the cannula from the implant site. FIG. 13C shows the delivery device 71 having been removed from the cephalad vertebral body 110 and anchorable member 30a now assuming its expanded configuration, with a radially expanded structure in the form of bowed struts. FIG. 13D shows the delivery device 71 still further withdrawn from the implant site, past the disc 201 within which a connector portion 50 is now visible spanning the transdiscal portion of the implant site. FIG. 13E shows the delivery device 71 partially removed from the caudal vertebral body and the proximal or second anchorable member now exposed but prior to expansion of the struts. FIG. 13F shows the delivery device 71 withdrawn to a point such that the proximal or second anchorable member 30b is released from the radial constraint that was being applied by the cannula 71, and its struts, now bowed, having expanded, the device 20 now visible, only the most proximal portion still engaged within cannula 71.

FIGS. 13B and 13H show a final step in the implantation, where the device is stabilized by the injection of a bone-filling composition 61, the composition having been described above. FIG. 13G shows a flowable cement 61 being injected through a delivery device 71, and the cement emerging from the device into the spaces within and surrounding the anchored members. FIG. 13H shows the delivery device having been completely removed from the implant site, and the device 20 transdiscally-implanted, anchored by expanded struts, and stabilized by the injected cement 61, now hardened in place.

In a second exemplary approach to implanting a transdiscal intervertebral body fusion device 20 (as seen fully assembled and deployed in FIG. 14G), the device is delivered not through the sidewall of one of the affected vertebrae, as above, but instead, it is delivered through the intervertebral space between the two adjacent vertebrae. FIGS. 14A-14H provide views of the deployment of a transdiscal intervertebral fusion device into two adjacent vertebral bodies through an intervertebral access channel, from which separately-formed but contiguously-joined cephalad and caudal channels are made into the interior of each and adjacent vertebral body, the device having an internal-external double body configuration, each body having four expandable struts. In a FIG. 14A shows a cannula 71 delivered drill 103 entering an intervertebral space and creating an entry into the caudal endplate of cephalad vertebral body 110 to form what will be a portion of a channel to receive an anchorable member of transdiscal intervetebral body fusion device.

FIG. 14B shows a cannula 71-delivered drill 103 entering an intervertebral space between compromised vertebrae 110, and creating an entry into the cephalad endplate of caudal vertebral body 110 to form a portion of a channel to receive an anchorable member of transdiscal intervetebral body fusion device. A portion of passageway 105a in the cepahalad vertebral body (from the drilling depicted in FIG. 14A) is now visible, the complementary portion of passage 105b now being drilled becomes visible in FIG. 14C.

FIGS. 14C and 14D show the delivery and deployment into an anchoring configuration of a first anchorable member into a first vertebral body (in this example, a vertebral body cephalad with respect to the adjacent vertebral body to which it will be fused). FIG. 14C shows the positioning of first anchorable member 30a′ into the cephalad vertebral body as it is being pushed from a cannula 71, by a push rod (not visible), as described above. The anchorable member 30a′ is still in the linear configuration in which it was being constrained while inside the radial confines of cannula 71, not having yet expanded, as will be seen in FIG. 14D. FIG. 14D shows the anchorable member self-expanding within the cephalad vertebral body 110 upon full emergence form the cannula 71.

FIG. 14E shows second anchorable member 30b implanted and deployed into the caudal vertebral body 110; by repeating the steps that implanted and deployed first anchorable member 30a (FIGS. 14C and 14D). In this embodiment of the device and method, the second anchorable member 30b includes a proximally-directed connector 50 within the interior of anchorable member 30b, which can be engaged and drawn out from the interior to engage the complementary anchorable member 30a to assemble a complete device. FIG. 14F shows a tool 63 (visible at the proximal opening of delivery device 71) having engaged the connector 50 and drawn it out of the interior of the second anchorable member, placing it so that it can engage the first anchorable member.

FIG. 14G shows the transdiscal intervertebral body fusion device after a bone filling composition 61 has been injected into the space within the expanded anchored members. The cement 61 has been injected into the lumen of the connector 50 through a side entry port (not shown) and flowed in both directions into the available space within the expanded struts of members 30a and 30b. Details and purpose of the cementing or bone-filling composition 61 has been described in detail above. Also shown in FIG. 14F is an example of how to draw anchorable members 30a and 30b closer together (as indicated by arrows), in order to position them optimally within the implant site. A gear mounted on the side of the connector is being turned by a complementary gear head at the distal end of a tool 63 (shown turning as indicated by arrow) that has been delivered to the site by a cannula 71, the turning of the gear results ultimately in the drawing together of the first and second anchorable members. Details of an exemplary embodiment of this mechanism is described further below and shown in FIG. 15. FIG. 14H shows the fully assembled transdiscal intervertebral body fusion device 20, now appropriately positioned by the adjustment of the relative position of the anchorable members and the connector as just described, and further stabilized by the now bone filling composition 61.

FIG. 15 depicts an embodiment of a transdiscal intervertebral body fusion system that is in the form of a kit 10, the kit including an Allen head tool 53 shown in a side view and a perspective view, a first anchorable member 30a and a second anchorable member 30b, two embodiments of a connector 50a and 50b, a delivery device 70, a container of a flowable bone filling composition 61, and an applicator, including a first rod 55 for engaging the first or distal anchor 30a, and a second rod 56 for engaging the second or proximal anchor 30b. The two rods of the delivery system may constrain the anchorable members from expanding during deployment. After delivery, one or both rods may be withdrawn, allowing anchorable members to contract and radially self-expand into anchoring configurations.

The delivery device 70 in this example has a distal threaded portion 72 that engages threads 58a on the first anchorable member 30a. The first anchorable member 30a has a connecting region (rod engaging feature 53a) that engages plug 59 on rod 55. The second anchorable member has a connecting region (rod engaging 53b) that engages plug 59 on rod 56. Rod 56 further has a stop bar 62 that meets the interior of the distal end of the second anchorable member and a plug mount 63 with plugs 59 that engage the proximal end of the second anchorable member. Rods 55 and 56 may both be considered embodiments of a length-constraining rod, which may constrain the length (in this case, preventing contraction) of an anchorable member, by engaging in a releasable way either or both the proximal or distal portion of an anchorable member in such a way that contraction of the member is prevented. The releasable-engagement means that interact between an anchorable member and a length-constraining rod may be of any suitable type. In the particular embodiments shown, the feature on the rods are male plugs that can rotate into female slots within the anchorable members, but the male-female orientation may be reversed in some embodiments, or more generally be of any suitable mechanism.

Two embodiments of a connector portion (50a and 50b) are shown in FIG. 15. Connector 50b is appropriate for use in implanting a device where a delivery device can engage a transdiscal intervertebral body fusion device in a linear manner, such as a proximally-positioned delivery device engaging the proximal or second anchorable member, as shown in FIGS. 13A-13H. Another exemplary approach to using a proximally-positioned (non-sleeved) delivery device is described in U.S. patent application Ser. No. 12/041,607 of Chirico et al., as filed on Mar. 3, 2008 (and incorporated by this reference), and depicted in FIGS. 16A-16O therein. Connector embodiment 50b is appropriate for use when a delivery or manipulating device engages the connector portion of a transdiscal intervertebral body fusion device from the side in order to adjust the relative distance between anchorable members, such as the implantation method detailed above and depicted in FIGS. 14A-14H.

Connector embodiment 50b has threaded portion 57a that engages threads 58a on first anchorable member 30a, and connector 50b also has threads 57b that engage threads 58b on second anchorable member 30b. Connector 50b further has an Allen head female feature 51b that engages the male head on Allen head tool 53. The threads 57a and 57b of the connector and their respectively engaging threads on the respective anchorable members are configured oppositely such that the connector 50 acts like a turnbuckle when turned by the Allen tool 53, and can thus pull the anchorable members together or extend them further apart. FIG. 16 shows an Allen wrench connector deployer 53 extending through the second anchorable member 30b to engage the connector at Allen female feature 51 within connector 50 and beginning to rotate the connector with respect to the two anchorable members, drawing them closer together, as indicated by the directional arrows.

Connector embodiment 50a has threaded portion 57a that engages threads 58a on first anchorable member 30a, and connector 50 also has threads 57b that engage threads 58b on second anchorable member 30b. Connector 50a further has a side-mounted Allen head female feature 51a that can be engaged by the male head on Allen head tool 53. Allen head female feature 51a is also rotatably engaged with two wedge-shaped gears 47 such that rotation of feature 51a in either direction rotates gears 47 in the opposite direction. The cogs of gears 47 each engage complementary cogs on the center-facing rims of cylinders 48. Cylinders 48 are rotatable portions of connector 50a, and rotate in place as driven by the rotating gears that engage cogs on their rim. The cylinders freewheel, and are held in place by an annular feature that fits into slot 82 at either end of connector 51a. Along the outer-facing portions of cylinders 48, their threads 57a and 57b engage complementary threads 58a and 58b on anchorable members 30a and 30b, respectively. Thus, in summary, as Allen head female feature 51a is rotated, gears 47 are rotated, cylinders 48 are rotated, and the rotation of threads 57a and 57b can draw anchorable members 30a and 30b either closer together, or further apart, depending on the direction of rotation of Allen head female feature 51a.

The foregoing description relates to an adjustment of a deployed transdiscal intervertebral body fusion device after it has been delivered and deployed into an anchorable configuration. The adjustment allows for fine tuning of the intervertebral distance, and in some instances may be adjusted even after the device has been implanted for a period of time.

Another type of adjustment was described earlier in which a mechanical assist may be applied to the struts of an anchorable member after the struts have already self-expanded to the degree that they can. FIG. 17 shows the first anchorable member being further expanded by a mechanical assist. The opposition rod 55 has been re-engaged (or has remained engaged) at the distal portion 59 of the first expandable member 30a, and the distal portion is being pulled proximally by rod 55. This is an optional step in the implantation of the device, and an analogous step may be taken with regard to the second or proximal anchorable member. Although the anchorable members are self-expanding, and expand to a preferred configuration when their expansion is unimpeded, when implanted in bone, such expansion can meet variable amounts of resistance, and not be able to independently attain their full degree or desired degree of expansion. For these reason, under some conditions, it may be desirable to mechanically assist in expansion of the struts of the anchoring configuration of an anchorable member. An analogous mechanical expansion step and a tool for such has been described in U.S. patent application Ser. No. 11/468,759.

Although the transdiscal intervertebral body fusion devices described herein typically include two anchorable (expandable) regions separated by a connector region, other variations are encompassed by this disclosure, including devices having more than two anchorable regions, which could be applied to the fusion of more than two vertebral bodies in a series. For example, a series of interconnected expandable regions could form a transdiscal intervertebral body fusion device. In addition, the connector regions could be formed of bendable, or rotatable material. In some variation the connector region or component is adjustable to shorten or lengthen the spacing between them without rotating them. For example, the connector region may be an interlocking telescoping region.

While the methods and devices have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A method of stabilizing two adjacent vertebral bodies, comprising:
forming a disc-traversing channel in adjacent first and second vertebral bodies, positioning a system for stabilizing the adjacent vertebral bodies in the channel, the system including:
a first anchorable member and a second anchorable member, each member having a central passageway, each member having a constrained non-anchoring configuration and a released anchoring configuration; and
a connector having a central passageway, the connector attachable to the proximal end of the first anchorable member and the distal end of the second anchorable member, such that the central passageways of the anchorable members and the connector form a continuous passageway; and
anchoring the first anchorable member within the first vertebral body and the second anchorable member within the second vertebral body.

2. The method of claim 1, wherein the channel is formed by percutaneously entering the second vertebral body, continuing through the disc, and terminating in the first vertebral body.

3. The method of claim 1, wherein the channel is formed by percutaneously entering a vertebral space between the adjacent vertebral bodies to create an access channel, forming a first portion of the disc traversing channel into the first vertebral body, and then forming a second portion of the disc traversing channel into the second vertebral body.

4. The method of claim 1 further comprising aligning the first vertebral body and the second vertebral body prior to forming the channel.

5. The method of claim 1 further comprising inserting an anchorable member into the channel in the constrained configuration.

6. The method of claim 1 further comprising constraining the anchorable members in the constrained configuration by preventing radial expansion with a sleeve.

7. The method of claim 1 further comprising releasing the anchorable members from the constrained configuration by ejection from a sleeve.

8. The method of claim 1 further comprising constraining the anchorable members in the constrained configuration by applying tension across the length of the members.

9. The method of claim 1 further comprising releasing the anchorable members from the constrained configuration by releasing tension from across the length of the members.

10. The method of claim 1 further comprising radially expanding a plurality of bowed struts from each anchorable member to anchor the first member and the second member within the first and second vertebral bodies respectively.

11. The method of claim 1 further comprising radially self-expanding a plurality of bowed struts from each anchorable member to anchor the first member and the second member within the first and second vertebral bodies respectively.

12. The method of claim 1 further comprising radially self-expanding a plurality of bowed struts from each anchorable member and then further expanding the struts mechanically to anchor the first member and the second member within the first and second vertebral bodies respectively.

13. The method of claim 1 further comprising simultaneously expanding the first and second anchorable members.

14. The method of claim 1 further comprising expanding the first anchorable member before expanding the second anchorable member.

15. The method of claim 1 further comprising exposing cutting surfaces on bowed struts forming the first and second anchorable members.

16. The method of claim 1 further comprising flowing a material through the continuous passageway.

17. The method of claim 16 further comprising hardening the flowable material to form a solid material.

18. The method of claim 1 further comprising flowing a material through the continuous passageway so that at least some material exits holes from the connector.

19. The method of claim 1 further comprising drawing the anchorable members closer together.

20. The method of claim 19, wherein drawing the anchorable members closer together includes rotating the connector.

21. A method of stabilizing adjacent vertebral bodies, comprising:
forming a channel in adjacent first and second vertebral bodies through adjacent endplate regions;
anchoring a first anchorable member within the channel in the first vertebral body;
anchoring a second anchorable member within the channel in the second vertebral body; and
flowing a material through a continuous central passageway formed through the first anchorable member, the second anchorable member and a connector between the first anchorable member and the second anchorable member.
